(19) United States
(12) Patent Application Publication
(10) Pub. No.: US 2011/0106141 A1
(43) Pub. Date: May 5, 2011

(54) ROBOT SYSTEM FOR ENDOSCOPIC TREATMENT

(76) Inventor: Toshio NAKAMURA, Hachioji-shi (JP)

(21) Appl. No.: 12/847,260

(22) Filed: Jul. 30, 2010

Related U.S. Application Data
(63) Continuation of application No. PCT/JP2010/050333, filed on Jan. 14, 2010.

(30) Foreign Application Priority Data
Mar. 24, 2009 (JP) 2009-071576

(51) Int. Cl. A61B 17/28

(52) U.S. Cl. 606/205

(57) ABSTRACT

A robot system for endoscopic treatment includes a gripping unit, a manipulator unit, a treatment instrument drive unit, a gripping force detection unit, a control unit. The gripping unit is inserted into a body cavity along with an endoscopic apparatus and grips desired living tissue using gripping members. The manipulator unit includes a multi-joint structure that enables bending and stretching. The treatment instrument drive unit pulls and pays out wires. The gripping force detection unit detects gripping force provided by the gripping members. The control unit instructs the treatment instrument drive unit to pull and pay out the wires for the gripping operation so that the gripping force detected by the gripping force detection unit falls within a predetermined range when the gripping unit in the gripping state moves.

### CROSS REFERENCE TO RELATED APPLICATIONS

This is a Continuation Application of PCT Application No. PCT/JP2010/050333, filed Jan. 14, 2010, which was published under PCT. Article 21(2) in Japanese.

This application is based upon and claims the benefit of priority from prior Japanese Patent Application No. 2009-071576, filed Mar. 24, 2009, the entire contents of which are incorporated herein by reference.

### BACKGROUND OF THE INVENTION

1. Field of the Invention

The present invention relates to a robot system for endoscopic treatment that adequately maintains a gripping state of a treatment instrument that is inserted into a body cavity.

2. Description of the Related Art

In general, there is known a robotic arm system for endoscopic treatment that remotely performs desired operation with respect to a treatment instrument that is inserted into an endoscope body or a forceps channel introduced in a body cavity.

In such a system, a master unit located outside includes an operation unit that is configured by a joystick or a plurality of joint members and rod members, the joint members and the rod members being alternately coupled with each other, and a master controller that converts movement of the operation unit into an electrical signal to be output as an operation signal. In a treatment instrument, a movable region at an end portion is coupled with, e.g., a pulley that is driven by a motor in a treatment instrument drive unit through a wire and the like. When this operation unit is operated, the pulley is rotated by the motor, and the wire is paid out or pulled. As a result, a biological sample (e.g., a lesioned part) can be moved in a desired direction in a gripped state, e.g., it can be gripped and pulled up by a treatment instrument inserted in a body cavity, e.g., an arm of a straight grasping forceps or a gripping unit provided at an arm end. As a representative example, there is known a multi-degree-of-freedom robotic treatment instrument having a soft portion. For example, Jpn. Pat. Appln. KOKAI Publication No. 2002-200091 discloses an example of such a technology.

### BRIEF SUMMARY OF THE INVENTION

According to an aspect of the invention, a robot system for endoscopic treatment, the system includes a gripping unit which is inserted into a body cavity along with an endoscopic apparatus and includes gripping members that are moved by a wire operation to grip desired living tissue; a manipulator unit which includes the gripping unit provided at an end thereof, has at least one degree of freedom, and includes a multi-joint structure that bends and stretches at a desired angle; a treatment instrument drive unit configured to pull and pay out wires which get the gripping unit performing a gripping operation and wires which get the manipulator unit moving; a gripping force detection unit which detects gripping force provided by the gripping members when the gripping unit grips the living tissue; and a control unit which instructs the treatment instrument drive unit to pull and pay out the wires for the gripping operation so that the gripping force detected by the gripping force detection unit falls within a predetermined range when the gripping unit gripping the living tissue moves.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a view showing a configuration of an entire robot system for endoscopic treatment in a first embodiment according to the present invention;

FIG. 2 is a view showing a configuration for controlling each component part and a flow of a detection signal, a control signal, and others in the first embodiment;

FIG. 3 is a flowchart for explaining wire tension control processing using a wire tension sensor in the first embodiment;

FIG. 4 is a view showing a configuration of an entire robot system for endoscopic treatment in a second embodiment according to the present invention;

FIG. 5A is a view showing a structural example of a gripping unit to which a pressure sensor is provided to detect gripping force in the second embodiment;

FIG. 5B is a view showing a structural example of a gripping unit in a monopolar high-frequency treatment instrument as a first modification of the gripping unit according to the second embodiment depicted in FIG. 5A;

FIG. 5C is a view showing a structural example of a gripping unit in a bipolar high-frequency treatment instrument as a second modification of the gripping unit according to the second embodiment depicted in FIG. 5A;

FIG. 6 is a view showing a configuration for controlling each component part and a flow of a detection signal, a control signal, and others in the second embodiment; and

FIG. 7 is a flowchart for explaining wire tension control processing using a pressure sensor in the second embodiment.

### DETAILED DESCRIPTION OF THE INVENTION

Hereinafter, embodiments according to the present invention will be described with reference to the drawings.

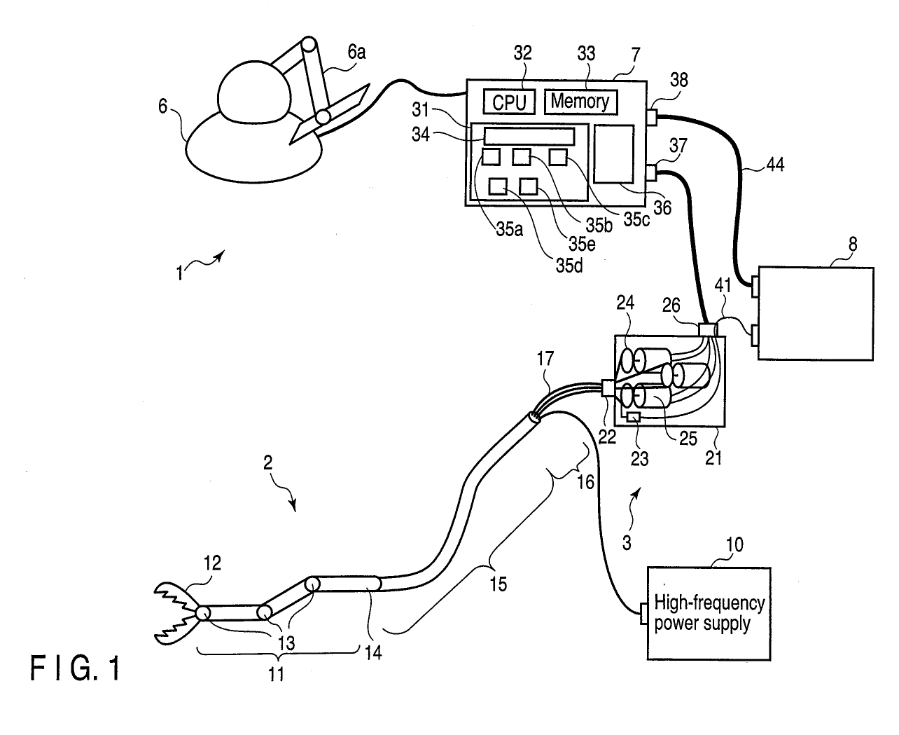

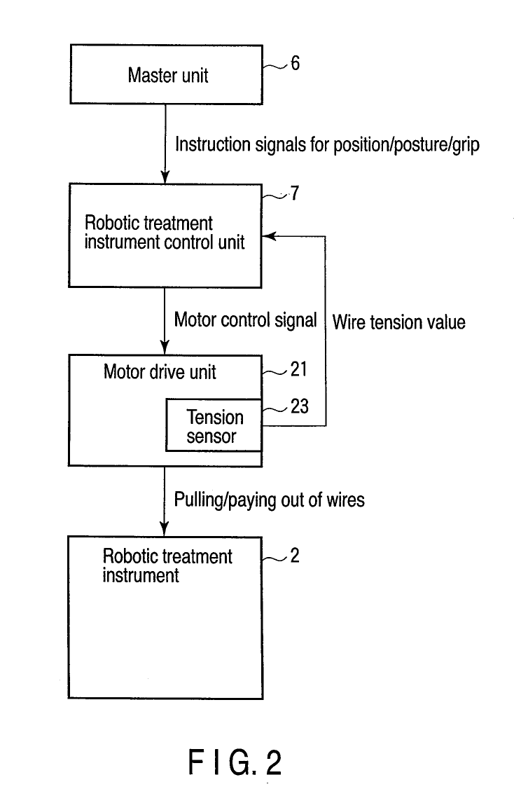

FIG. 1 is a view showing a configuration of an entire robot system for endoscopic treatment in a first embodiment according to the present invention. FIG. 2 is a view showing a configuration for effecting control in each constituent unit in the robot system for endoscopic treatment and a flow of a detection signal, a control signal, and others.

The robot system for endoscopic treatment according to this embodiment comprises a robotic treatment instrument having a multi-joint structure driven by a plurality of wires, and a wire tension sensor provided to the wires that drive a manipulator unit and a gripping unit, the wire tension sensor detecting gripping force, the robot system executing gripping control processing based on an obtained wire tension value (the gripping force).

This robot system for endoscopic treatment roughly comprises a controller 1 including a master unit 6, a robotic treatment instrument 2, and a treatment instrument drive unit 3. This robotic treatment instrument 2 is a master/slave multi-joint electric treatment instrument that follows an operation of the master unit 6 which is an instruction input device. Furthermore, the gripping unit may have a function of a bipolar high-frequency treatment instrument that holds living tissue and applies high-frequency electrical power from a high-frequency power supply 10 to perform high-frequency treatment.

Although the explanation will be given as to the configuration where the master unit 6 in this embodiment issues a bend instruction for a manipulator unit 11 and an open/close instruction (gripping instruction) for a gripping unit 12, an operating portion that issues the gripping instruction may be provided separately from the master unit 6. Moreover, the master unit 6 may be configured integrally with an operation unit in the endoscope body so that one operating portion can issue an instruction for the robotic treatment instrument 2 and an instruction for the endoscope body.

The controller 1 comprises the master unit 6 which is configured to instruct a position, a posture, and gripping of the later-described robotic treatment instrument 2, a robotic treatment instrument control unit 7 that controls the robotic treatment instrument 2, and a tension sensor signal processor 8 which is configured to detect a gripping state.

The master unit 6 includes an operating portion 6a in which a plurality of joint members and rod members are alternately coupled with each other and a non-illustrated master controller that converts movement of the operating portion 6a into an electrical signal and outputs the converted signal as an operation signal. Moreover, in the operating portion 6a is provided a non-illustrated switch which is configured to instruct operations for effecting an opening/closing operation of the gripping unit 12 to grip living tissue and release the living tissue.

The robotic treatment instrument control unit 7 include an operation setting unit 31 that is configured to set various kinds of settings for the robotic treatment instrument 2; a CPU 32 that executes processing for each later-described sensor signal and various kinds of arithmetic operations, and outputs a control signal to each constituent unit in the system; a memory 33 that stores programs required for driving, obtained arithmetic operation results, and communication data; a motor driver 36 that drives and controls a motor 25 in a later-described motor drive unit 21 based on the control signal; a motor driver communication unit 37 that is configured to communicate with the later-described motor drive unit 21; and a tension sensor communication unit 38 that is configured to communicate with a later-described tension sensor signal processor 8.

The operation setting unit 31 includes operation switches 35a to 35e that is configured to set various kinds of settings and a display panel 34 that displays contents of an operation instructed by a user.

The robotic surgical instrument 2 includes the manipulator unit 11 having the multi-joint structure driven by the wires, the gripping unit 12 that is provided at an end of the manipulator unit 11 to grip living tissue (lesioned part as a treatment target), and a soft sheath portion 15 which is inserted into a non-illustrated endoscope channel and can move forward and backward. It is to be noted that the robotic treatment instrument 2 is not necessarily restricted to a usage pattern that it is inserted into the endoscope to be used, and it may be utilized separately from the endoscope. Also, the gripping unit 12 is a bipolar high-frequency treatment instrument having a configuration that two gripping members which are formed of a conductor or have opposed electrodes provided thereto, the treatment instrument opening/closing to grip living tissue and performing high-frequency treatment.

The manipulator unit 11 is provided at a distal end of the soft sheath portion 15, and is formed of at least two rod portions 14 and at least one joint portion 13 that couples the rod portions 14 with each other, resulting in the manipulator unit 11 having at least one degree of freedom. For example, the manipulator unit 11 has one degree of freedom in a vertical direction. Preferably, the manipulator unit 11 is configured in such a manner the plurality of rod portions 14 and the plurality of joint portions 13 are alternately coupled to provide different rotation surfaces of joints, so as to have six degrees of freedom in XYZ directions, a rotating direction, a yawing direction, and a pitch direction, resulting in that the manipulator unit 11 can freely bend to lift up and move the gripping unit 12. Each of the two gripping members (jaws) of the gripping unit 12 and the respective joint portions 13 are connected with wires 17 inserted in the manipulator unit 11, the sheath portion 15, and an external connecting portion 16. When these wires 17 are pulled and paid out from the external connecting portion 16, the gripping members can be opened/closed, and the respective joint portions 13 can be bent and stretched at desired angles. This opening/closing operation and the bending/stretching operation are carried out by the treatment instrument drive unit 3.

For example, if each rod portion 14 has a cylindrical shape, a connecting configuration of the rod portions 14 and the wires 17 in this embodiment is achieved by coupling each rod portion 14 with the joint points 13 at two cylinder opening ends in the horizontal direction to allow the bending operation, disposing an end of each wire 17 to each of two cylinder opening ends in the vertical direction orthogonal to the horizontal direction, and coupling the other end of the same with a manipulator (e.g., a pulley pivotally supported by a motor). When bending the rod portion 14 with respect to the joint portion 13, one of the wires 17 is pulled and the other of the wires 17 is paid out, resulting in that the rod portion 14 bends upward (or downwards) around a coupling portion between the rod portion 14 and the joint portion 13. Of course, this embodiment is not restricted to such a multi-joint structure, and a generally known multi-joint structure can be applied to this embodiment.

The treatment instrument drive unit 3 includes a motor drive unit 21 with a plurality of motors which are individually controlled. The motor drive unit 21 includes a plurality of pulley 24 coupled with the wires 17, a plurality of motors 25 which axially support the respective pulleys 24, wire tension sensors 23 which measure tensile force of respective wires 17, and a motor driver communication unit 26 which communicates with the robotic treatment instrument control unit 7.

In this embodiment, one pulley 24 is coupled with a set of wires 17 connected with the two movable gripping members of the gripping unit 12 or respective joint portion 13 through a wire coupling portion 22. Furthermore, although the description has been given on the example where a combination of the motor and the pulley is a manipulator that drives the wires 17, a combination of a servomotor and a bar-like coupling tool may also be adopted. In this case, two wires are coupled with both ends of the bar-like coupling tool and fixing a central portion of this tool to the servomotor. Moreover, electric hydrodynamic drive based on a combination of a hydraulic piston, an electric pump, and a valve can also be considered. In this case, wires are respectively coupled with the two hydraulic pistons, and the valve is opened/closed to pull/pay out the wires.

The motor drive unit 21 further includes a non-illustrated motor which rotates the robotic treatment instrument 2 on a longitudinal axis, a non-illustrated motor which moves forward and backward the robotic treatment instrument 2 (the sheath portion 15), and a non-illustrated encoder which measure a rotating angle of each motor. It is to be noted that the wire tension sensor 23 may be arranged in the wire coupling portion 22. As the wire tension sensor 23, a strain gauge that can detect a slight change in length of each wire 17 in a longitudinal axis direction is preferable. A wire tension value measured by the wire tension sensor 23 is output to the tension sensor signal processor 8 through a cable 41 and further supplied to the robotic treatment instrument control unit 7 through a cable 44.

The component parts and a flow of signals in the robot system for endoscopic treatment will now be described with reference to FIG. 2.

The master unit 6 output an instruction signal which instructs a position and a posture with respect to the manipulator unit 11, and an instruction signal which instructs gripping and releasing living tissue with respect to the gripping unit 12 to the robotic surgical instrument control unit 7 by operation of the operating portion 6a. The robotic treatment instrument control unit 7 supplies a motor control signal to the motor drive unit 21 to drive the corresponding motor 25 in response to the instruction signal received from the master unit 6. Based on this driving, the pulley 24 pivotally supported by the motor 25 rotates, and then pull and pay out the wires 17 so that movement and a change in posture of the manipulator unit 11 coupled with the wires 17 or gripping and releasing of the gripping unit 12 are carried out. When the gripping unit 12 is in a gripping state, the tension sensor 23 is allowed to detect a wire tension value of the wires 17 coupled with the gripping members. This wire tension value is fed back to the robotic treatment instrument control unit 7. Based on such feedback control, the living tissue can be held by the gripping unit with the same gripping force, or the manipulator unit 11 can be maintained in the same posture (the gripping unit is maintained at the same position).

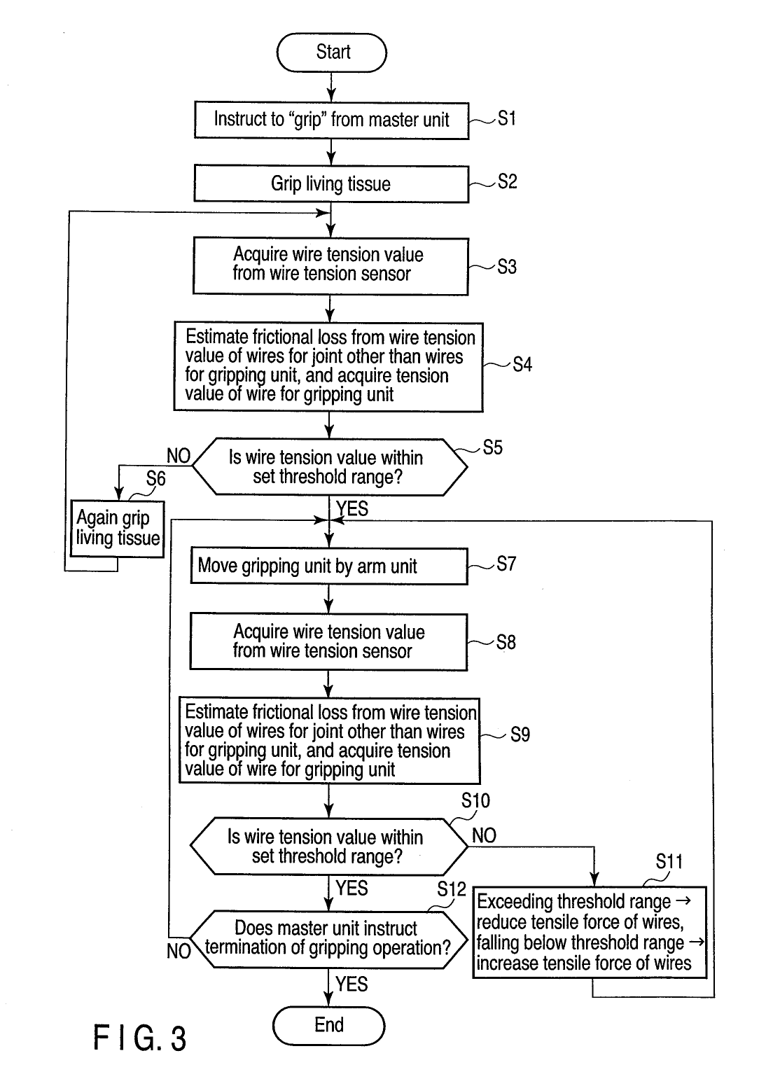

Wire tension control processing of the gripping unit 12 when movement is achieved with the manipulator unit 11 by using the wire tension sensor in the robot system for endoscopic treatment will now be described with reference to a flowchart depicted in FIG. 3.

An operator (a surgeon) operates the master unit 6 to instruct the robotic treatment instrument control unit 7 to grip desired living tissue with the gripping unit 12 inserted in a body cavity (step S1). Based on this instruction, the robotic treatment instrument control unit 7 drives the corresponding motor 25 in the motor drive unit 21 to rotate the pivotally supported pulley 24 so that the wires 17 coupled with the pulley 24 are pulled and paid out, thereby gripping the living tissue by using the gripping unit 12 (step S2).

At the time of gripping, the robotic treatment instrument control unit 7 acquires all wire tension values from the wire tension sensor 23 connected to the wires 17 (step S3). The robotic treatment instrument control unit 7 estimates a loss due to friction based on the wire tension values of the wires 17 coupled with the rod portions (or the other joint portions) excluding the wires 17 coupled with the gripping unit 12 in these wire tension values, and subtracts the loss (frictional force) caused due to the friction obtained based on the wire tension values that are utilized to drive the rod portions (the joint portions) from the wire tension value of the wires 17 that perform gripping (step S4).

It is to be noted that although the example where the tension sensor is directly disposed to the wires to measure tensile force has been described in this embodiment, the tension sensor in structural requirements of the present invention is not restricted thereto. For example, a method of acquiring tensile information from information of the utilized actuator is also an aspect as the tension sensor. For example, when a motor is used as the actuator, current measuring unit or current calculating unit functions as the tension sensor. Further, when a shaped-memory alloy (SMA) is used for the actuator, temperature information acquiring unit for the SMA functions as the tension sensor.

Step S4 will be further explained. The wire tensile force and the gripping force of the end gripping units have a correlative relationship based on a proportional relation. That is, when the wire tensile force is large, the gripping force increases in proportion to the wire tensile force. However, when the joints or the soft portions are bent, it is considered that a loss of strength due to friction of a non-illustrated coil pipe through which the wires 17 run and the wires 17 occurs. Thus, a correlation of a bending angle of any other joint portion with a wire tension value of the wires 17 that bends the joint portion and loss due to friction of the wires 17 that drive the gripping unit 12 at this moment is acquired in advance, the wire tension value of the wires 17 and loss due to friction of the wires 17 being in proportion to a magnitude of bending of each joint portion excluding the gripping unit 12. Then, when measuring the gripping force from the wire tension value, an accurate wire tension value of the wires 17 that operate the gripping unit 12 can be obtained by subtracting the loss due to the friction that is obtained by utilizing the previously acquired correlation from the wire tension value for driving the gripping unit 12. In this manner, the accurate gripping force can be obtained.

Then, the robotic treatment instrument control unit 7 compares the wire tension value obtained in step S4 with a predetermined threshold range and determines whether the wire tension value is within the threshold range (step S5). In this determination, if the wire tension value is out of the threshold range (NO), the robotic treatment instrument control unit 7 determines that the gripping unit 12 is not appropriately gripping the living tissue, controls the gripping unit 12 to again grip (step S6), and returns to step S3 to check the wire tension value. On the other hand, if the wire tension value is within the threshold range (YES), the robotic treatment instrument control unit 7 shifts to the next step. At the time of performing treatment, for example, if living tissue in a gripped lesioned part is increased due to exfoliation, the manipulator unit 11 is driven to move the gripping unit 12 while the gripping unit 12 grips the living tissue in order to visually confirm a treatment region by the surgeon (step S7). During this movement, the robotic treatment instrument control unit 7 acquires wire tension values from all the wire tension sensor 23 of the wires 17 (step S8), estimates and subtracts a loss due to friction (frictional force) from the obtained wire tension values like step S4, and acquires tensile force of the wires 17 which operate the gripping unit 12 (step S9).

Subsequently, the robotic treatment instrument control unit 7 compares the wire tension value obtained in step S9 with a predetermined threshold range and determines whether the wire tension value is within the threshold range (step S10). This determination is occasionally made in a time-sharing manner, i.e., made at appropriately set time intervals. If the wire tension value is out of the threshold range (NO) in this determination, the robotic treatment instrument control unit 7 determines that the gripping state of the gripping unit 12 is no loner adequate. The robotic treatment instrument control unit 7 drives and controls the motor 25 to reduce the wire tensile force if the wire tension value exceeds the threshold range in this determination and increase the wire tensile force if the wire tension value falls below the threshold range in the same (step S11). On the other hand, if the wire tension value is within the threshold range in the determination in step S10 (YES), the robotic treatment instrument control unit 7 determines that the gripping unit 12 keeps normally gripping the living tissue, and determines whether an instruction for termination (including temporary pause or suspension) of the gripping operation is issued from the master unit 6 in response to end of the treatment (step S12). If there is no instruction for termination of the gripping operation (NO), the robotic treatment instrument control unit 7 returns to step S7 to continue movement of the gripping unit 12. On the other hand, when there is the instruction for termination of the gripping operation (YES), the robotic treatment instrument control unit 7 terminates the series of processing.

As described above, according to the robot system for endoscopic treatment of this embodiment, the wire tension sensor is provided to the wires which drive the manipulator unit and the gripping unit of the robotic treatment instrument having the multi-joint structure. When the gripping unit is moved by driving the manipulator unit, the wires are controlled to be paid out and pulled until the wire tension value falls within the predetermined threshold range based on the wire tension value obtained from the wire tension sensor so that a normal gripping state in the gripping unit can be maintained. As a result, when the gripping unit which is in the gripping state with bending the joints in the manipulator unit is moved to a desired position, a change in gripping force due to a difference between wire path lengths or unevenness in gripping force due to friction between the wires and the coil pipe can be eliminated, slack or unnecessary pulling force is not produced in the wires, thereby maintaining the normal gripping state of the living tissue.

Furthermore, if the robotic treatment instrument is a bipolar high-frequency treatment instrument, the instrument can grip the living tissue as a treatment target with appropriate gripping force, and perform high-frequency treatment to a body mucosal membrane, e.g., sealing and cauterizing a blood vessel or a vessel bundle present inside.

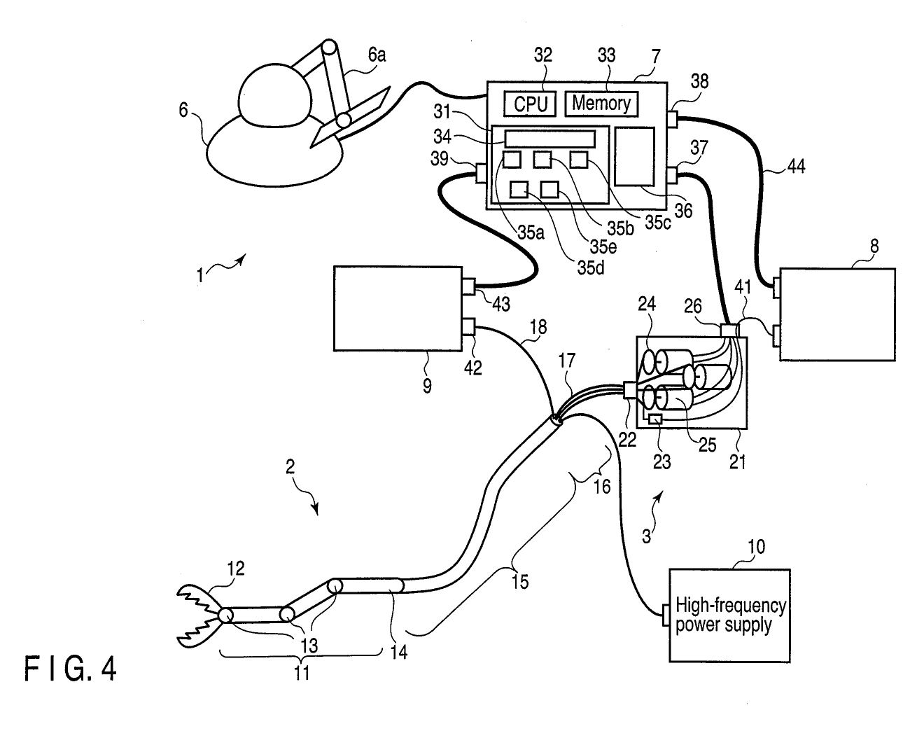

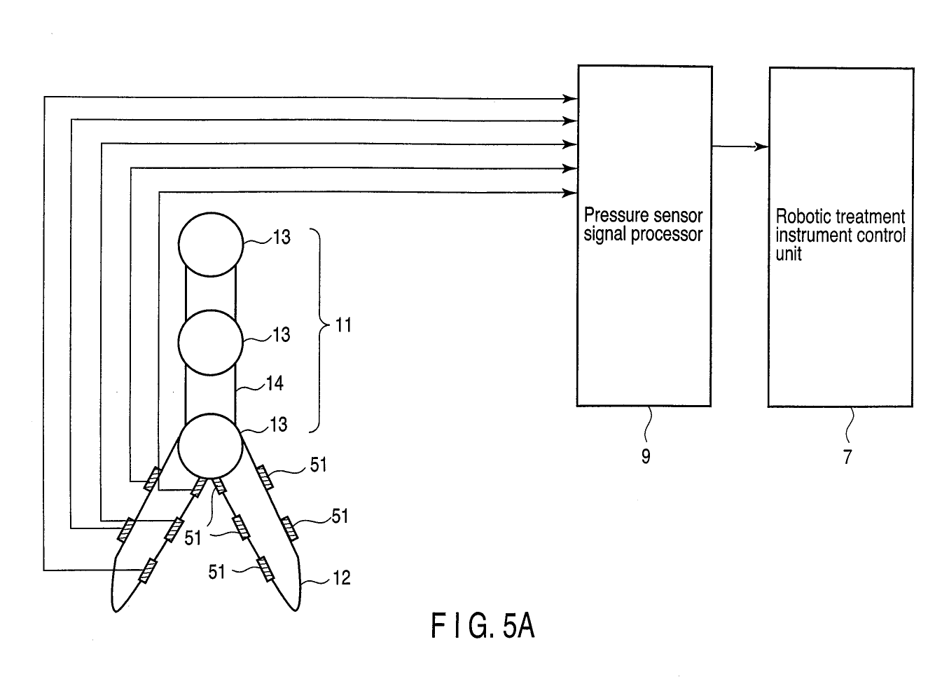

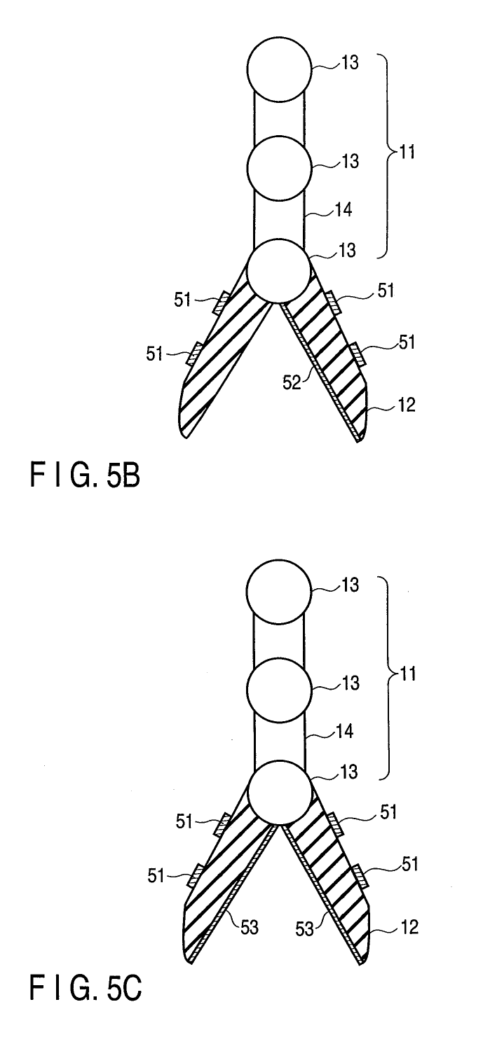

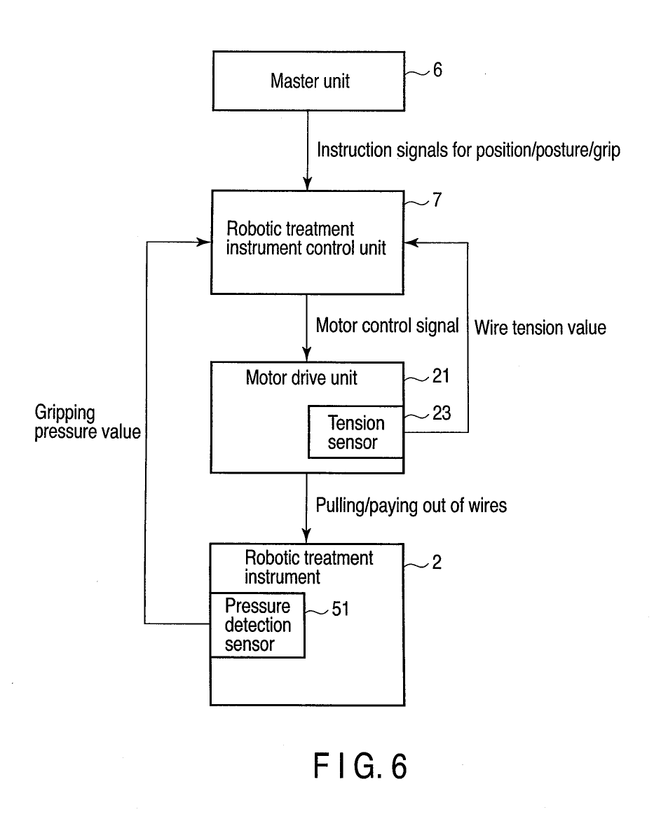

FIG. 4 is a view showing an entire configuration of the robot system for endoscopic treatment in a second embodiment according to the present invention. FIG. 5A is a view showing a structural example of a gripping unit having pressure sensors provided thereto to detect gripping force in the robot system. FIG. 6 is a view showing a configuration for controlling each component parts and a flow of a detection signal, a control signal, and others in the robot system for endoscopic treatment. Like reference numbers denote component parts in this embodiment equivalent to those shown in FIGS. 1 and 2 in the first embodiment, thereby omitting a detailed description thereof.

The robot system for endoscopic treatment according to this embodiment includes a robotic treatment instrument having a master/slave multi-joint structure which is driven by the same kinds of wires 17 as those in the first embodiment. Pressure sensors 51 configured to detect gripping force are provided to a gripping unit 12 of this robotic treatment instrument, and gripping control processing are carried out based on the gripping force value obtained when living tissue are gripped. It is to be noted that a wire tension sensor 23 is likewise provided with respect to a manipulator unit 11 in this embodiment, and the wire tension sensor does not necessarily have to be provided with respect to the gripping unit 12.

A control unit 1 includes a master unit 6, a robotic treatment instrument control unit 7, a tension sensor signal processor 8, and a pressure sensor signal processor 9 configured to detect a gripping state of the gripping unit 12. The robotic treatment instrument control unit 7 likewise includes an operation setting unit 31, a CPU 32, a memory 33, a motor driver 36, a tension sensor communication unit 38, a pressure sensor communication unit 39 configured to communicate with the pressure sensor signal processor 9, and a motor drive unit communication unit 37. The operation setting unit 31 includes operation switches 35a to 35e and a display panel 34. Furthermore, in the pressure sensor signal processing unit 9 includes a receiving communication unit 42 that receives gripping pressure values from the pressure sensors 51 and a transmitting communication unit 43 that transmits gripping pressure values to the pressure sensor communication unit 39.

The gripping unit 12 depicted in FIG. 5A is a bipolar high-frequency treatment instrument configured in such a manner that two gripping members having conductors or electrodes open and close to grip living tissue and perform high-frequency treatment. One or more pressure sensors 51 configured to detect gripping force for gripping the living tissue are disposed on front and back surfaces (a gripping surface with which the living tissue comes into contact and a non-gripping surface) of each of these gripping members. The pressure sensors 51 may be uniformly disposed to the front and back surfaces of the gripping unit 12 or may be densely arranged mainly in a region of each gripping surface with which the living tissue comes into contact. The necessary number of pressure sensors 51 may be arranged while appropriately considering a shape, a gripping state, and others of each gripping member.

The gripping unit 12 outputs gripping pressure values (sensor output signals) from the pressure sensors 51 each suggesting a living tissue gripping situation (whether a gripping state is adequately maintained) to the robotic treatment instrument control unit 7.

These gripping pressure values from the pressure sensors 51 are acquired by the pressure sensor signal processing unit 9 in a time-sharing manner, i.e., acquired at predetermined time intervals while gripping, e.g., a living mucosal membrane by the gripping members. Moreover, when strain gauges are used as the pressure sensors 51, since the strain gauges detect slight deformation of the gripping unit, the strain gauges do not necessarily have to be provided on the inner surfaces of the gripping members, and they may be provided on the outer surfaces of the same. The gripping pressure values measured by the pressure sensors 51 are amplified in the pressure sensor signal processor 9 and input to the robotic treatment instrument control unit 7. Additionally, a value of each encoder is transmitted to the robotic treatment instrument control unit 7. It is to be noted that, in the plurality of pressure sensors, the pressure sensors 51 which are not in contact with the living tissue are also present. In this case, a lower limit threshold value is set in advance, and it can be determined that the pressure sensor 51 which detect the pressure value not greater than the threshold value is not gripping the living tissue, and the output pressure value can be canceled to invalidate the value.

Further, FIGS. 5B and 5C are views showing first and second modifications of the gripping unit 12 in the second embodiment.

The first modification shown in FIG. 5B is a monopolar high-frequency treatment instrument. A gripping unit 12 of the treatment instrument the gripping unit opening and closing is formed of an insulator, and has a configuration provided an electrode portion 52 on a entire region of one gripping surface which may possibly come into contact with living tissue. In regard to pressure sensors 51, a plurality of pressure sensors 51 are arranged on outer sides of the gripping unit 12, i.e., non-gripping surfaces (which do not come into contact with living tissue).

The second modification shown in FIG. 5C is a bipolar high-frequency treatment instrument. A gripping unit 12 of the treatment instrument the gripping unit opening and closing is formed of an insulator, and has a configuration that provided electrode portions 53 are individually provided on entire regions of both gripping surfaces which may possibly come into contact with living tissue. In regard to pressure sensors 51, the plurality of pressure sensors 51 are arranged on outer sides like the first modification.

These first and second modifications can obtain the same functions and effects as those of the first embodiment. Moreover, since the electrode portions are provided on the entire surfaces of the gripping surfaces which come into contact with living tissue, contact can be assuredly achieved irrespective of a size of the living tissue.

Component parts and a flow of signals in the robot system for endoscopic treatment in the second embodiment will now be described with reference to FIG. 6. It is to be noted that the description is simplified in regard to parts equivalent to those in FIG. 2.

The master unit 6 outputs an instruction signal for the manipulator unit 11 and an instruction signal for the gripping unit 12 based on an operation of a surgeon to the robotic treatment instrument control unit 7. The robotic treatment instrument control unit 7 drives the corresponding motor 25 in the motor drive unit 21 in response to these instruction signals and thereby rotates the pivotally supported pulley 24. Based on this rotation, the wires 17 are pulled and paid out so that movement or a change in posture of the manipulator unit 11 coupled with the wires 17 or gripping or releasing of the gripping unit 12 coupled with the wires 17 is carried out.

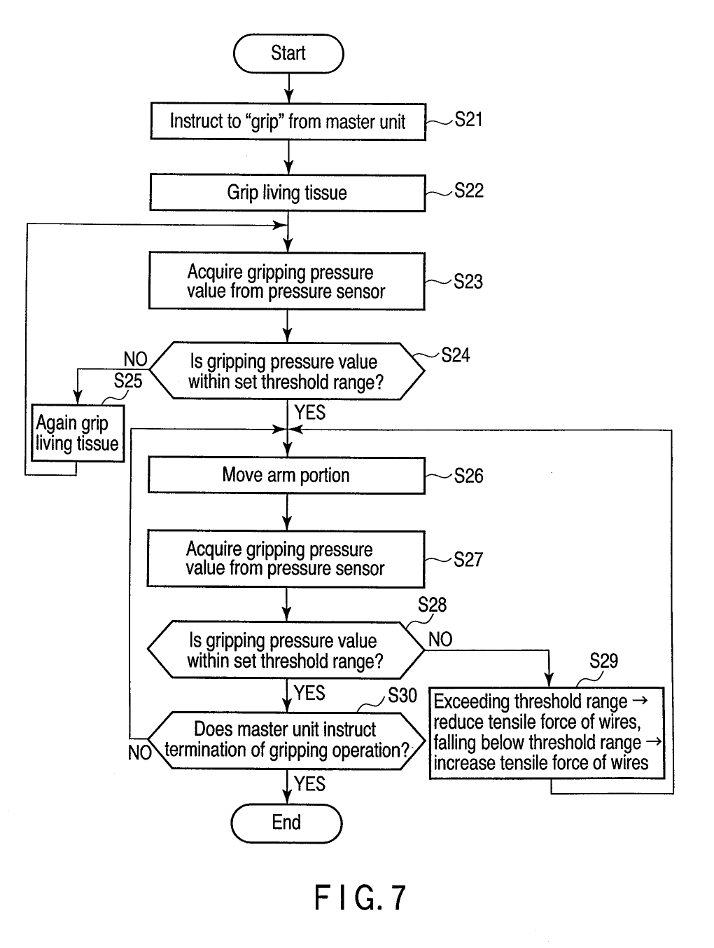

When the gripping unit 12 is in a gripping state, gripping pressure values from the pressure sensors 51 are acquired by the pressure sensor signal processor 9, subjected to signal processing, and then fed back to the robotic treatment instrument control unit 7. Based on such feedback control, the same gripping force in the gripping unit can be sustained, or the manipulator unit 11 can be maintained in the same posture (the gripping unit can be maintained at the same position). Such sustention will be described later in conjunction with FIG. 7.

As described above, at the same time as the actual gripping operation is performed, gripping pressure values detected by the pressure sensors 51 provided in the gripping unit 12 are subjected to signal processing in the pressure sensor signal processor 9, input to the robotic treatment instrument control unit 7, and compared with a threshold value having a predetermined given range (width). In this comparison, when the gripping pressure value from each pressure sensor 51 is within the threshold range, the gripping state based on the current wire tensile force is maintained. On the other hand, when the gripping pressure value is out of the threshold range, the wire tensile force is adjusted until this value falls within the threshold range.

Wire tensile control processing of the gripping unit 12 using the pressure sensors in the robot system for endoscopic treatment when the gripping portion is moved through the manipulator unit 11 will now be described with reference to a flowchart depicted in FIG. 7.

An operator (surgeon) operates the master unit 6 to instruct the robotic treatment instrument control unit 7 to grip desired living tissue by using the gripping unit 12 inserted in a body cavity (step S21). Based on this instruction, the robotic treatment instrument control unit 7 drives the corresponding motor 25 in the motor drive unit 21 and thereby rotates the pivotally supported pulley 24 to pull and pay out the wires 17 coupled with the pulley 24, thus gripping the living tissue by using the gripping unit 12 (step S22). At the time of gripping, the robotic treatment instrument control unit 7 acquires gripping pressure values from the pressure sensors 51 provided to the gripping unit 12 (step S23).

Then, the robotic treatment instrument control unit 7 compares each gripping pressure value with a predetermined threshold range and determines whether the gripping pressure value is within the threshold range (step S24). In this determination, when the gripping pressure value is out of the threshold range (NO), the robotic treatment instrument control unit 7 determines that the gripping unit 12 is not appropriately gripping the living tissue, operates the gripping unit 12 to again grip the living tissue (step S25), and returns the processing to step S23 to check the gripping pressure value. On the other hand, when the gripping pressure value is within the threshold range (YES), the robotic treatment instrument control unit 7 advances the processing to the next step.

At the time of performing treatment, for example, when living tissue in a gripped lesioned part is increased due to exfoliation, the manipulator unit 11 is driven to move the gripping unit 12 while the gripping unit 12 grips the living tissue in order to visually confirm a treatment region by the surgeon (step S26). At the time of this movement, the robotic treatment instrument control unit 7 acquires gripping pressure values from he pressure sensors 51 at predetermined time intervals (step S27), and occasionally determines whether the gripping pressure values are within the threshold range by comparing the gripping pressure values with the threshold range (step S28).

In this determination in step S28, when each gripping pressure value is out of the threshold range (NO), the robotic treatment instrument control unit 7 determines that the gripping state in the gripping unit 12 is no longer adequate. The robotic treatment instrument control unit 7 drives and controls the motor 25 to reduce wire tensile force when the gripping pressure value exceeds the threshold range in this determination and increase the wire tensile force when the gripping pressure value falls below the threshold range (step S29). On the other hand, if the gripping pressure value is within the threshold range (YES) in this determination in step S28, the robotic treatment instrument control unit 7 determines that the gripping unit 12 keeps normally gripping the living tissue and determines whether an instruction for termination (including temporary pause or suspension) of the gripping operation is issued from the master unit 6 in response to end of the treatment (step S30). When there is no instruction for termination of the gripping operation (NO), the robotic treatment instrument control unit 7 returns the processing to step S26 to continue movement of the gripping unit 12. On the other hand, when there is the instruction for termination of the gripping operation (YES), the robotic surgical instrument control unit 7 terminates the series of processing.

As described above, according to the robot system for endoscopic treatment of this embodiment, the pressure sensors are provided to the gripping unit of the robotic treatment instrument with the multi-joint structure, and control is effected to pay out or pull the wires based on a gripping pressure value obtained from each pressure sensor when the gripping unit is moved by drive of the manipulator unit until the gripping pressure value falls within the predetermined threshold range so that the normal gripping state in the gripping unit can be maintained. Based on this control, even if the gripping unit which is in the gripping state with the manipulator unit bending the joints is moved to a desired position, a change in gripping force due to a path length changing of the wires inserted inside or unevenness in gripping force due to friction between wires and coil pipes, resulting in gripping unit maintaining the normal gripping state for living tissue without producing slack or unnecessary pulling force in the wires.

Moreover, in the first embodiment according to the present invention, the description has been given as the robotic treatment instrument in the robot system for endoscopic treatment is the bipolar high-frequency treatment instrument as an example, the gripping unit having configuration with the bipolar high-frequency treatment instrument. When the robotic treatment instrument is used as the bipolar high-frequency surgical instrument, the gripping unit is formed by using an insulating member such as ceramics or a resin so that electrical short circuit can be prevented from occurring between the two gripping members. The electrodes are provided to the gripping units of these gripping members, high-frequency electrical power is applied to these electrodes at the time of gripping, and high-frequency surgery of sealing and cauterizing a part of a body cavity, a blood vessel, and others is performed. The robotic treatment instrument according to the first embodiment can be readily applied to the monopolar high-frequency treatment instrument. In this case, one of the two gripping members is formed of a conductor or an electrical insulating member (e.g., ceramics or zirconia) with an electrode provided thereto, and the other is formed of an electrical insulating member. A P-plate is attached to a patient to use the treatment instrument like a regular monopolar high-frequency treatment instrument. Of course, an instrument other than the P-plate may be used as long as it is an instrument which functions as an opposed electrode.

Also, in the second embodiment according to the present invention, the robotic treatment instrument in the robot system for endoscopic treatment can be readily configured to have a function of the bipolar high-frequency treatment instrument or the monopolar high-frequency treatment instrument. When providing the function of the bipolar high-frequency treatment instrument to the second embodiment, the pressure sensors provided to the gripping unit is prevented from being damaged due to heat generated by application of high-frequency power. Specifically, since the pressure sensors cannot be arranged on portions of the gripping unit that grip living tissue (the gripping surfaces), the pressure sensors should be provided on the non-gripping surfaces. Therefore, when strain gauges are used as the pressure sensors 51, since the strain gauges can detect slight deformation of the gripping unit, the strain gauges can be provided on the non-gripping surfaces. When applying to the monopolar high-frequency treatment instrument, like the first embodiment described above, one of the gripping members is formed of a conductor or an electrical insulating member with an electrode provided thereto, the other of the same can be formed of an electrical insulating member, and an instrument (e.g., a P-plate) that functions as an opposed electrode can be arranged.

It is to be noted that the robot system for endoscopic treatment includes the following aspects.

(1) A medical robot system characterized by comprising:

a gripping unit which opens and closes by a wire operation from a surgeon's front side;

gripping force detection unit configured to detect gripping force in the gripping unit;

controlling unit configured to control various kinds of actuators based on inputs from a master unit operated by the surgeon and various kinds of sensors including the gripping force detection unit; and

a manipulator unit having at least one degree of freedom,

wherein the controlling unit controls outputs to the actuators so that an output value from the gripping force detection unit falls within a given threshold range.

(2) The medical robot system in the term (1) characterized in that the gripping force detection unit is a pressure sensor provided at an end of the gripping unit.

(3) The medical robot system in the term (1) characterized in that the gripping force detection unit is a wire tension sensor disposed to the wires.

(4) The medical robot system in the term (1) characterized in that the gripping unit is a monopolar or bipolar high-frequency treatment instrument.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1.-10. (canceled)

11. A robot system for endoscopic treatment comprising:
a gripping unit configured to be inserted into a body cavity along with an endoscopic apparatus wherein a desired living tissue is to be gripped by gripping members configured to move by a wire operation;
a manipulator unit having at least one degree of freedom or more and including a multi joint structure configured to bend at a desired angle or stretch wherein the gripping unit is provided at a distal end;
a treatment instrument drive unit configured to perform a pulling and a paying out of wires for a gripping motion of the gripping unit and moving a region of the multi joint structure;
a gripping force detection unit configured to detect gripping force in the gripping members when the gripping unit grips the living tissue; and
a control unit configured to instruct the treatment instrument drive unit on the wire operation of the gripping members so that the gripping force detected by the gripping force detection unit falls within a range of a predetermined threshold, when the gripping unit in a gripping state is moved.

12. The robot system for the endoscopic treatment according to claim 11,
wherein the treatment instrument drive unit is composed of:
pulleys wherein one ends of each of pairs of wires are coupled to each of the gripping members of the gripping unit and the movable region of the multi joint structure of the manipulator unit and the other ends of each of the pairs of wires are coupled to each of the pulleys; and
motors wherein each of the motors pivotally supports each of the pulleys and is configured to drive by control of the control unit, and
the treatment instrument drive unit is configured to open and close the gripping members and bend at the desired angle and stretch the movable region of the multi joint structure by pulling and paying out the wires accompanied with rotation of the pulleys.

13. The robot system for the endoscopic treatment according to claim 11,
wherein the gripping force detection unit is composed of a wire tension sensor configured to detect the gripping force based on tensile force of the wire coupling to the gripping members of the gripping unit.

14. The robot system for the endoscopic treatment according to claim 11,
wherein the gripping force detection unit is composed of one pressure sensor or more arranged at the gripping members of the gripping unit and configured to output a gripping pressure value when the living tissue is gripped.

15. The robot system for the endoscopic treatment according to claim 11,
wherein the manipulator unit is configured to freely bend at six degrees of freedom of XYZ directions, a rotating direction, a yawing direction and a pitch direction by coupling rod members and joint members with each other alternately.

16. The robot system for the endoscopic treatment according to claim 13,
wherein the robot system for the endoscopic treatment includes:
electrodes wherein each of the electrodes is provided with a gripping surface of each of the two gripping members and the two gripping members are formed of an insulating material; and
a high-frequency power supply configured to apply high-frequency power to the electrodes when the living tissue is gripped, and
the robot system for the endoscopic treatment has a function as a bipolar high-frequency treatment instrument configured to perform a high-frequency treatment including sealing and cauterizing with respect to the living tissue.

17. The robot system for the endoscopic treatment according to claim 13,
wherein the robot system for the endoscopic treatment further comprises:
an electrode provided on a gripping surface of one of the two gripping members wherein the two gripping members are formed of an insulating material;
an opposed electrode arranged near the electrode; and
a high-frequency power supply configured to apply high-frequency power to the electrode when the living tissue is gripped, and
the robot system for the endoscopic treatment has a function as a monopolar high-frequency treatment instrument configured to perform a high-frequency treatment including sealing and cauterizing with respect to the living tissue.

18. The robot system for the endoscopic treatment according to claim 14,
wherein the robot system for the endoscopic treatment further comprises:
electrodes wherein each of the electrodes is provided with a gripping surface of each of the two gripping members, the two gripping members are formed of an insulating material and the pressure sensor is arranged on a non-gripping surface of the gripping unit; and
a high-frequency power supply configured to apply high-frequency power to the electrodes when the living tissue is gripped, and
the robot system for the endoscopic treatment has a function as a bipolar high-frequency treatment instrument configured to perform a high-frequency treatment including sealing and cauterizing with respect to the living tissue.

19. The robot system for the endoscopic treatment according to claim 14, wherein the pressure sensor is arranged on a non-gripping surface of the gripping unit, the robot system for the endoscopic treatment is composed of:
an electrode provided on a gripping surface of one of the two gripping members wherein the two gripping members are formed of an insulating material;
an opposed electrode arranged near the electrode; and
a high-frequency power supply configured to apply high-frequency power to the electrode when the living tissue is gripped, and
the robot system for the endoscopic treatment has a function as a monopolar high-frequency treatment instrument configured to perform a high-frequency treatment including sealing and cauterizing with respect to the living tissue.

20. The robot system for the endoscopic treatment according to claim 11,
wherein the robot system for the endoscopic treatment is a master/slave robot system comprising a master unit which is an instruction input device for remotely operating the gripping unit and the manipulator unit.

21. A robot system for endoscopic treatment comprising:
gripping means for being inserted into a body cavity along with an endoscopic means wherein a desired living tissue is to be gripped by gripping member means for moving by wire means operation;
manipulator means having at least one degree of freedom or more and including multi joint means for bending at a desired angle or stretching wherein the gripping means is provided at a distal end;
treatment instrument drive means for performing a pulling and a paying out of wire means for a gripping motion of the gripping means and moving a region of the multi joint means;
gripping force detection means for detecting gripping force in the gripping member means when the gripping means grips the living tissue; and
control means for instructing the treatment instrument drive means on the wire means operation of the gripping member means so that the gripping force detected by the gripping force detection means falls within a range of a predetermined threshold, when the gripping means in a gripping state is moved.
